(19) United States
(12) Patent Application Publication
(10) Pub. No.: US 2014/0012353 A1
(43) Pub. Date: Jan. 9, 2014

(54) Light Therapy for Increasing CD34+ Cell Count in Peripheral Blood

(71) Applicant: Marvin A. Prescott, Los Angeles, CA (US)

(72) Inventor: Marvin A. Prescott, Los Angeles, CA (US)

(21) Appl. No.: 14/024,560

(22) Filed: Sep. 11, 2013

Related U.S. Application Data
(63) Continuation-in-part of application No. 13/079,627, filed on Apr. 4, 2011.
(60) Provisional application No. 61/322,748, filed on Apr. 9, 2010.
(60) Provisional application No. 61/797,310, filed on Dec. 4, 2012.
(60) Provisional application No. 61/797,309, filed on Dec. 4, 2012.

(51) Int. Cl. A61N 5/06

(52) U.S. Cl. CPC A61N 5/0613
USPC 607/89; 607/88

(57) ABSTRACT

Techniques for elevating CD34+ cell level in peripheral blood using light are disclosed herein. In one example, a light generating device is positioned to a body part of a patient, and a beam of light generated by the light generating device is directed to the body part of the patient to increase CD34+ cell level in the peripheral blood of the patient. In various embodiments, the beam of light is direct to a body part of the patient that is CD34+ stem/progenitor cell and/or precursor cell that give rise to CD34+ stem/progenitor cell rich. In various embodiments, the body part is the abdomen of the patient rich in adipose tissue. In various embodiments, the light beam has one or more wavelength in the range of 400 to 1300 nm. In various embodiments, the light beam is a low intensity light beam of generated by a power of 1-12 mW.

### CROSS REFERENCE TO OTHER APPLICATIONS

This application is a continuation in part of co-pending U.S. patent application Ser. No. 13/079,627 (Attorney Docket No. DRP10301) entitled Method of Managing Metabolic Syndrome filed Feb. 4, 2011 which in turn claims priority from U.S. Provisional Patent Application No. 61/322,748 filed on Apr. 9, 2010; this application is a continuation in part of a co-pending U.S. patent application Ser. No. 13/12,983 (Attorney Docket No. MARVIN001) entitled Light Therapy for Treating or Managing Diabetes and Metabolic Syndrome filed on Mar. 6, 2012; this application is a non-provisional application of prior filed U.S. Provisional Application No. 61/797,310 (Attorney Docket No. MARVIN002+) entitled Light Therapy for Treating or Managing Alzheimer's Disease filed on Dec. 4, 2012; this application is a non-provisional application of prior filed U.S. Provisional Application No. 61/797,309 (Attorney Docket No. MARVIN003+) entitled Light Therapy for Treating or Reversing Endothelial Dysfunction filed on Dec. 4, 2012; wherein the entirety of all U.S. priority applications is herein incorporated herein by reference for all purposes.

### BACKGROUND OF THE INVENTION

CD34 molecule is a glycosylated transmembrane protein, cells that express CD34 protein are termed CD34+ cells. The CD34+ cells are typically pluripotent stem and progenitor cells that can differentiate into various types of cells including all types of blood cells, blood vessel endothelial cells, cardiocytes, smooth muscle cells (See Yeh, et al., Circulation 108:2070-73 (2003)). However not all CD34-expressing cells are stem and progenitor cells, endothelial cells express CD34 protein but do not seem to possess the inherent expansive potential of stem and progenitor cells. Bone marrow, cord blood and adipose tissues are main sources of CD34+ cells and CD34− stromal cells that give rise to CD34+ cells.

Small amounts of CD34+ cells normally circulate in the peripheral blood. The circulating CD34+ cells are believed to have originated from the bone marrow and/or the adipose tissue, and consist of hematopoietic stem/progenitor cells that can differentiate into all blood cell types and endothelial stem/progenitor cells that take part in angiogenesis and vaculogenesis in neovascularization and revascularization. Increased mobilization of CD34+ cells from bone marrow and/or adipose tissue into the peripheral blood can also occur under certain clinical conditions, for example, CD34+ cells were observed to be elevated in the peripheral blood after stroke, myocardial infarction and cerebral ischemia.

Upon mobilization, the CD34+ cells tend to home to injured and ischemic tissues to promote healing by for example participating in building blood vessels either through their direct incorporation into newly developing blood vessel or through their secretion of angiogenic growth factors that stimulate local peri-endothelial vascular development. The ability to effectively mobilize CD34+ cells into blood (e.g., from bone marrow and/or adipose tissue) is thus important for proper tissue maintenance and healing. The beneficial effect of CD34+ cells in the repair of various ischemic and injured tissues has been well documented. Blood CD34+ cell count is found to be negatively correlated to occurrences of various disease states and positively correlated with their prognosis, for example, the numbers of circulating CD34+ cells in healthy individuals are found to be strongly inversely proportional to the occurrence of cerebral infarction (Taguchi A et al. Circulating CD34-positive cells provide an index of cerebrovascular function. Circulation. 2004 June 22; 109(24):2972-5). The peripheral blood mononuclear CD34+ cells level after stroke was found to be positively correlated to neurological and functional recoveries of stroke patient. (Dunac A et al. Neurological and functional recovery in human stroke are associated with peripheral blood CD34+ cell mobilization. J. Neurol. 2007 March; 254 (3):327-32). Atherosclerosis is found to be associated with reduced numbers and dysfunction of endothelial progenitor cells (EPCs) which is a subset of circulating CD34+ cells (Bielak L F et al. Circulating CD34+ cell count is associated with extent of subclinical atherosclerosis in asymptomatic Amish men, independent of 10-year Framingham risk. Clin Med Cardiol. 2009 May 27; 3:53-60).

CD34+ cells have been used in various therapeutic applications, such as healing of diabetic wounds, myocardial infarction, stroke, and retinopathy by participating in and promoting angiogenesis and/or vaculogenesis in neovascularization and/or revascularization. CD34+ cells used are typically harvested from a variety of tissue sources including bone marrow, cord blood, adipose tissue and peripheral blood, either from the patient him/herself or from a donor. The harvested CD34+ cells are often expanded ex vivo before being introduced back into patients.

Although only a small percentage of cells in the peripheral blood are CD34+ cells, peripheral blood is potentially an important source of CD34+ because it is the most accessible and least invasive way for harvesting CD34+ cells and new technologies are now being developed to effectively harvest CD34+ cells from the peripheral blood. The CD34+ cell count in the peripheral blood will influence the ease of CD34+ stem and progenitor cell harvesting from the peripheral blood. The higher the peripheral blood CD34+ cell count, the easier it is to harvest the CD34+ cells from the peripheral blood.

Various factors have been found to influence the effective mobilization of CD34+ cells into blood and thus the CD34+ cell counts in peripheral blood. Health factors such as smoking and alcohol abuse negatively affect CD34+ cells. Advanced age and certain disease states such as metabolic syndrome and diabetes may hamper the functionality of CD34+ cells and mobilization of CD34+ cells into blood. Exercise has been known to improve mobilization of CD34+ cells into peripheral blood. Certain pathophysiologic ischemic conditions promote mobilization of CD34+ cells, for example myocardial ischemia and peripheral ischemia are known to stimulate CD34+ cell mobilization. Certain pharmaceutical and biopharmaceutical mobilization agents such as granulocyte-colony stimulating factor (G-CSF), granulocyte-macrophage colony-stimulating factor (GM-CSF) have been used to increase CD34+ mobilization.

Since we cannot always rely on pathophysiologic ischemic conditions to enhance CD34+ cell mobilization, exercise may not be suited or preferred by all patients, and the pharmaceutical or biological mobilization agents can be highly toxic and may not be tolerated by some individuals, other alternative methods of effectively mobilizing CD34+ cells into peripheral blood that are safe and gentle on individual patients are needed.

It has come to the inventor(s) attention that hyperbaric oxygen improves mobilization of CD34+ cells through nitric oxide dependent mechanism (Aicher A et al. Essential role of endothelial nitric oxide synthase for mobilization of stem and progenitor cells. Nat Med. 2003; 9:1370-1376). Additionally, nitric oxide was found to support the proliferation and mobilization of the endothelial committed progenitor subpopulation in bone marrow (Ozuyaman B et al. Nitric oxide differentially regulates proliferation and mobilization of endothelial progenitor cells but not of hematopoietic stem cells. Thromb Haemost 2005 October: 94(4):770-2). It is also known that low power light has been found to increase nitric oxide (Mitchell U H et al. Low-level laser treatment with near-infrared light increases venous nitric oxide levels acutely: a single-blind, randomized clinical trial of efficacy. Am J Phys Med Rehabil. 2013 February; 92(2):151-6). The invention therefore addresses the question whether low power light increases mobilization CD34+ cells into blood.

### BRIEF DESCRIPTION OF THE DRAWINGS

Various embodiments of the invention are disclosed in the following detailed description and the accompanying drawings.

FIG. 1 illustrates an example light-generating device for mobilizing CD34+ cells.

FIG. 2 illustrates example inner electronic components of the light-generating device of FIG. 1.

FIG. 3 illustrates an example external light-generating device with contoured body configured to fit a patient's body contour.

FIG. 4 illustrates an example method for mobilizing CD34+ cells with a portable light generating device.

FIG. 5 illustrates an example embodiment for mobilizing CD34+ cells.

FIG. 6 illustrates an example embodiment for mobilizing CD34+ cells.

FIG. 7 illustrates another example embodiment for mobilizing CD34+ cells.

FIG. 8 illustrates another example embodiment for mobilizing CD34+ cells.

FIG. 9 illustrates an example embodiment for ex vivo expansion of CD34+ cells and administration of the expanded CD34+ cells to a patient.

### DETAILED DESCRIPTION

The invention can be implemented in numerous ways, including as a method; a process; a therapy; an apparatus; a system; a device (external and/or implantable); and a composition of matters. A detailed description of one or more embodiments of the invention is provided below along with accompanying figures that illustrate the principles of the invention. The invention is described in connection with such embodiments, but the invention is not limited to any embodiment. In the specification, the various implementations of the invention may be referred to as techniques. In general, the order of the steps of disclosed processes may be altered and one or more steps of disclosed processes may be omitted within the scope of the invention.

Techniques for mobilizing CD34+ cells are disclosed herein. In various embodiments, a light generating device is positioned to a body part of a patient rich in CD34+ cells and/or precursor cells that give rise to CD34+ cells such as stromal cells, and a beam of light generated by the light generating device is directed to the body part of the patient to cause increased level of CD34+ cells to enter into peripheral blood stream. In various embodiments, the body part includes a body area rich in adipose tissue, such as the abdominal area, thigh, buttocks, and upper arms of the patient. Adipose tissue is known as a rich reservoir of CD34+ stem/progenitor cells and precursor cells that give rise to CD34+ stem/progenitor cells. In various embodiments, the CD34+ cells were mobilized from the adipose tissue of the body part of the patient to which the light generating device is directed to. In various embodiments, the light beam has one or more wavelength in the range of 400 to 1300 nm. In various embodiments, the light beam has wavelength in the red and near infrared region. Light in this wavelength are known to penetrate tissue well and free from carcinogenic effect of lights in other wavelength region. In various embodiments the light beam has a wavelength in the range of 610 to 900 nm. In various embodiments, the light beam has a wavelength in the range of 800 to 900 nm. In various embodiments, the light beam is a low intensity light beam generated by for example a power of 1-12 mW using for example a laser diode or light emitting diode. It is conceivable a lower intensity light beam may be used, provided the light beam can penetrate the body to reach the targeted tissue and the exposure time is long enough that the patient receives adequate light therapy dose. It is also conceivable the light beam is a higher intensity light beam, provided that the exposure time is short and the patient is not over dosed. In various embodiments, the low intensity light beam is generated by a power of 1-5 mW. In various embodiments, the low intensity light beam is generated by a power of 1-3 mW. In various embodiments, the low intensity light beam is generated by a power of 1-2 mW. In various embodiments, the low intensity light beam is generated by a power of 2-3 mW. In various embodiments, the light beam is directed to a body part of the patient according to a prescribe regimen.

The inventor(s) herein hypothesize that low intensity light placed over a body part rich in CD34+ stem/progenitor cells and/or precursor cells that give rise to CD34+ stem/progenitor cells, such as adipose tissue of a patient/person, increases nitric oxide (NO) production and bioavailability, which in turn mobilizes CD34+ cells into peripheral blood. In particular, when a low intensity light is shone over the abdominal adipose tissue of a patient, it preferentially increases the production of CD34+ endothelial stem/progenitor cells in the adipose tissue and their mobilization into peripheral blood. It should be understood that the above proposed mechanisms are not limiting to the present invention, one or more alternative mechanisms may be at work in conjunction to the above proposed mechanisms or instead of the above proposed mechanisms.

The increased blood CD34+ stem/progenitor cell counts in the peripheral blood can help to improve tissue repair and regeneration. Particularly, the increased blood CD34+ endothelial stem/progenitor cells in the peripheral blood home to ischemic and damaged tissues to help tissue healing and repair by participating in neovascularization and revascularization. When the increased blood CD34+ stem/progenitor cell count in the peripheral blood is applied to various clinical situations, it helps to reduce disease risk and improve outcome in various clinical situations such as in myocardial infarction, stroke, diabetic foot ulcer, diabetic retinopathy, peripheral vascular disorder, myocardial ischemia, cerebral ischemia.

The increased blood CD34+ stem/progenitor cells in the peripheral blood can also make CD34+ cell harvesting more feasible and easier. The harvest CD34+ cells can be expanded or multiplied ex vivo in cell culture medium. The ex vivo expanded CD34+ cells can be administered back to a patient to help with tissue healing/repair in various clinical situations such as in myocardial infarction, stroke, diabetic foot ulcer, diabetic retinopathy, peripheral vascular disorder, myocardial ischemia, and cerebral ischemia.

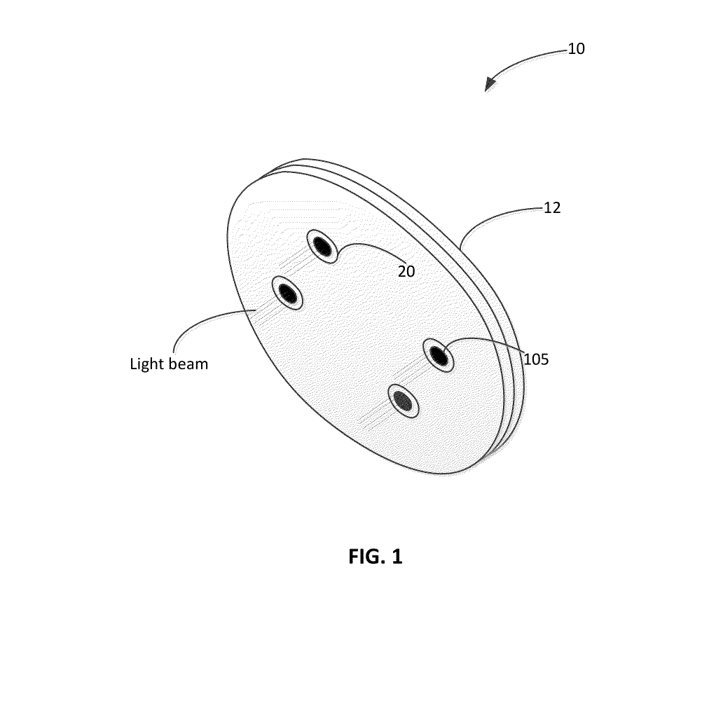

FIG. 1 illustrates an example light-generating device 10 for mobilizing CD34+ cells. One or more light-generating devices 10 may be positioned to a patient's body, for example, to a specific body part or body region, and operated according to a prescribed light regimen. The specific body part or region may be rich in CD34+ cells and/or precursor cells that give rise to CD34+ cells. The specified body part or region may be a region that rich in underlying adipose tissue, such as stomach (visceral) areas, thigh areas, buttocks areas, upper arm areas. Adipose tissues are known to be rich in CD34+ cells and/or precursor cells that give rise to CD34+ cells. The light-generating devices 10 may be positioned to (e.g. attached via bandage, strap) the external surface of the skin of the specific body region or may be implanted to the specific body region, for example in the subcutaneous tissue of the specific body region.

Light-generating device 10 includes a body 12. The material used to form the body 12 may depend on whether the light generating device is configured as an external device or implantable device.

In various embodiments, the light generating device is an implantable device that is configured to be implanted inside a patient's body, such as in a subcutaneous space, and in such embodiments, the body should be made of biocompatible materials that can exist in harmony with the patient's body without causing deleterious changes when implanted. Important criteria of biocompatible material include for example that the material being nontoxic and eliciting little or no immune response from the patient's body. Example biocompatible materials that can be used to form the body include titanium metal and glass ceramics. In various embodiments, the device may be embedded in various scar prevention materials, such as collagen and microvascular construct (MVC) to minimize scar formation around the device when it is implanted. In various embodiments, the body may be coated with a layer of nanoporous ceramic membrane to prevent protein build up around the implanted device. The use of implantable light generating device for managing or treating diabetes or metabolic syndromes has the advantage of ensuring patient compliance and minimize or eliminate injuries, particularly retinal injuries caused by laser light if laser light is used as the light source.

In various embodiments, the light generating device is an external device. In various embodiments, the device may be a wearable and portable device that is configured to be positioned over the skin of a patient, and in such embodiments, the biocompatibility of the material for forming the body may not be important, so it can be made of a variety of materials as long as the material provides adequate protection to the various components of the light-generating device. For example, the light generating device can be made of p-cell or plastozote or lux plastic to make it non-flexible, light-weight, and portable. Alternatively, the body of the light-generating device may be made of flexible materials, such as molded silicone, to render the device flexible, light-weight, and portable. Alternatively, the device may not be a portable device, for example, the device may be placed in a caregiver's office (e.g., physician's office) and the patient may be required to travel to the caregiver's office to be treated to ensure proper use and safety.

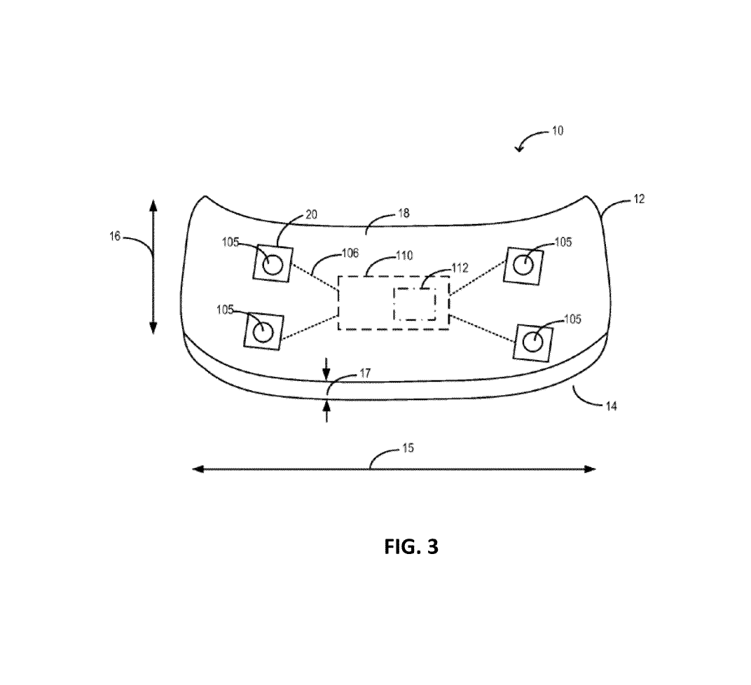

The body 12 may be designed as a casing within which are housed a light source, a power source, a programmable controller, and/or other sensors of the device 10. The device may be designed as virtually any shape. As a non-limiting example, the device 10 is depicted in FIG. 1 as having an oval body 12. In another non-limiting example, the device is substantially rectangular body 12 having a length 5 inches, a width 2.5 inches, and a thickness ¼ inch. The dimensions of the device may be adjusted based on the desired application of the device. When the device is configured as an external device, the body 12 may be contoured to reduce discomfort when the device is attached to the patient's body (as shown in FIG. 3). The contour 14 of the device may be adjusted to conform to the body part to which it is to be attached. For example, the device may be contoured differently if designed for use on the stomach or visceral adipose area than when designed for use on the thigh or upper leg area. Alternatively, the body of the device may be made of flexible material so the device can conform to the contour of the body part by virtue of its flexible nature.

The body 12 of the device may include openings 20, formed therein, to accommodate light produced by an underlying light source 105. The openings may be provided with clear windows to protect the underlying light source from dust, oils, dirt, etc. The clear windows may be formed of glass, plastic, titanium or other suitable materials. Alternatively, the clear windows may be formed of the same material as the body of the device. As such, this would allow the windows to be co-molded with the body 12. Further, if the device 10 is configured as an implantable device, the material used to form the openings 20 also needs to be biocompatible material.

In some embodiments, device 10 may be encased in a pre-formed, sterilized, cover (not shown) held in place by a friction fit over body 12. In embodiments where the device is configured as an external device, the cover may further include a cushioning agent to cushion the device when attached to the patient's body, the cover may also be disposable. The disposable or sterilized nature of the cover ensures that only sterile surfaces contact the patient's body and treatment area. When included, the cover may also have a plurality of openings that are aligned with the (underlying) openings 20 of body 12. In embodiments where the device is configured as an external device, body 12 may further include strap hooks (not shown) for receiving a strap for supporting the device. In other embodiments, the device may be attached to a target area of the patient's body using flexible bandage.

In the depicted example, device 10 includes four light sources 105 distributed over the surface area of the device. The light sources 105 may be configured to generate a low power light. As such, the light sources may be configured to be flexible or non-flexible, based on the flexible nature of the device. As non-limiting examples, the light sources may include any combination of lasers (or laser diodes), light emitting diodes (LEDs), and/or infrared devices. Based on the nature of the light source, the light beam generated by the device may have a wavelength ranging from 400 nm to 1300 nm. In various embodiments, the light beam includes a wavelength in the range of 400 nm to 1300 nm. In various embodiments, the light beam includes a wavelength in the red or near infrared range. In various embodiments, the light beam includes a wavelength in the range of 600 to 900 nm. In various embodiments, the light beam includes a wavelength in the range of 800 to 900 nm. In various embodiments, the light beam includes a wavelength in the range of 840 to 860 nm. In various embodiments, the light beam consists of light having wavelength ranging from 400 nm to 1300 nm. In various embodiments, the light beam consists of light in the red or near infrared range. In various embodiments, the light beam consists of light having wavelength ranging from 600 to 900 nm. In various embodiments, the light beam consists of light having wavelength ranging from 800 to 900 nm. In various embodiments, the light beam consists of light having wavelength ranging from 840 to 860 nm.

In some embodiments, device 10 may further include a focusing lens (not shown) for focusing the low power light generated by the light source onto the body of the patient. However in alternate embodiments, such as where the light source is capable of generating a collimated and focused beam, additional focusing lenses may not be required.

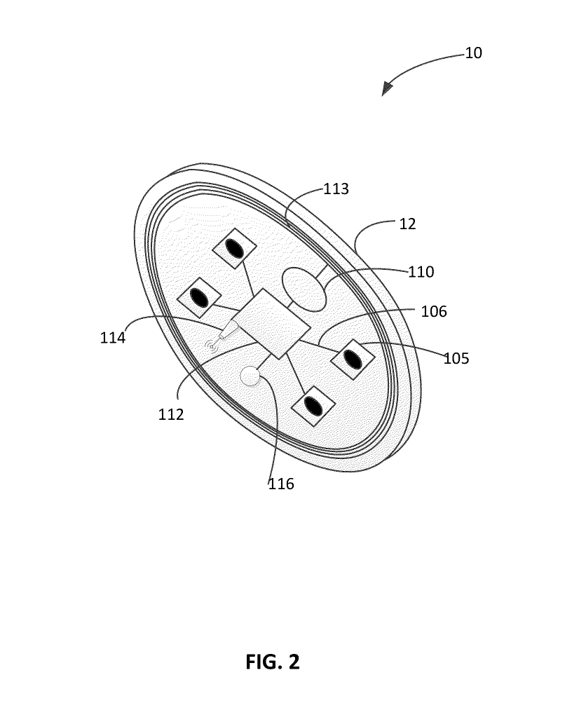

FIG. 2 illustrates the inner electronic components of the light-generating device 10 of FIG. 1. Light sources 105 may be interconnected inside the device by flat wire connectors 106. The flat wire connectors 106 may lead to a power source 110 and a programmable controller 112. Power source 110 powers the light sources 105. Various small and portable power sources can be used, such as one or more batteries. The batteries may include, for example, one or more wafer thin lithium polymer batteries, or rechargeable nickel-metal hydride batteries. In one example, when rechargeable batteries are included in power source 110, the batteries may be recharged when device 10 is not enabled, or not attached to the patient's body. In various embodiments, a wireless charging coil (113) is included to wirelessly recharge the battery. In this way, the device power source 110 may be recharged wirelessly through an external recharging system while implanted in a patient's body. In various embodiments, an adaptor is included to allow the battery to be charged using an external power source via a wired connection.

Programmable controller 112 may include a control circuit printed onto a circuit board. As such, programmable controller may be configured with code for selectively enabling the light source 105 of the device 10 according to the prescribed light regimen. The prescribed light regimen may be determined by the patient's healthcare provider based on one or more patient parameters such as body mass index, body weight, and blood CD34+ cell count. The light regimen may include a plurality of emission periods during which low power light is directed towards the specific body part. Each of the plurality of emission periods may be separated from each other by interval periods during which no low power light is generated. This intermittent way of shining light on the patient may help to save battery power and extend wearable time of the device without the need for recharging and reduce battery size. Example light regimens are discussed below.

Specifically, the programmable controller may selectively enable the light source (for example, by connecting light source 105 to power source 110) for a period of time and then selectively disable the light source for a period of time immediately following (for example, by disconnecting light source 105 from power source 110).

Device 10 may optionally include a wireless receiver (114) for wirelessly communicate with external computing devices. In various embodiments, the wireless receiver is an integral part of the control circuit 112. In various embodiments, computer instructions including for example prescribed light regimen can be communicated to the device 10 from an external computing device via the wireless receiver. The various patient parameters measured through the various sensors can be communicated to an external computing device via the wireless receiver.

In embodiments where the device 10 is configured as an external device, it may optionally include a proximity sensor (not shown) for determining the proximity of the device to the patient's body. The proximity sensor may be configured as a chip included in the body 12 of the device. In one example, the proximity sensor may be activated when the device 10 is within a threshold distance of the patient's body (e.g., when the device is placed on the waist/visceral area of the patient). The proximity sensor may communicate that the sensor (or the device) is within a threshold distance of the patient's body to the healthcare provider (e.g., via wireless communication). In this way, with the help of the proximity sensor, a doctor may be able to monitor the wearing of the device and make sure that the patient is wearing the device as prescribed. In various embodiments, the proximity parameter provided by the proximity sensor may be used to automatically turn on/off the light source. In this way, the light source is turned on only when the device is close to a solid surface (e.g., the body part) to minimize the chance laser light generated by the source does not accidentally cause retinal damage to the patient, a caregiver, or someone standing nearby.

The device may further include one or more patient parameter sensors 116 for measuring one or more patient parameters of the patient, such as glucose, cholesterol (HDL & LDL), adiponectin, liver enzyme (SGPT & SGOT), triglyceride, lymphocyte, monocyte, blood pressure, blood oxygen levels, etc. Based on the parameters to be measured, appropriate parameter sensors may be included in the device. In various embodiments, the sensors 116 may include interfaces for measuring the patient parameters. In various embodiments, the patient parameters may be measured via sensors external to the device and communicated to the device via a communication interface.

Since the body 12 of the device houses the light sources, power sources, programmable controller, and various sensors, support posts may be optionally included within body 12 to enhance the rigidity of the device, as well as to provide spacing within the body to delineate the internal components.

In one embodiment (as depicted), light sources 105 are low-power lasers, such as vertical-cavity surface-emitting lasers (VCSELs) having a nominal power output of 2.6 mW and a wavelength in the range of 600-850 nm (herein, a wavelength of approximately 750 nm). The power output of the lasers may range from 2.6 to 10 mW. At this operating level, sufficient power can be provided for the treatment of metabolic syndrome with an extended life of the power source.

As such, VCSELs comprise semiconductor lasers which emit a collimated beam normal to the surface of the semiconductor substance. The semiconductor typically comprises aluminum arsenide (AIAs) or gallium arsenide (GaAs), or a combination thereof. Each VCSEL has a self-contained, high-reflectivity minor structure forming a cavity to produce the collimated beam. Due to the ability of VCSELs to produce a collimated beam, additional focusing lenses may not be required in device to focus the beam when VCSELs are the light source. Additionally, VCSELs provide reduced size and low power consumption benefits. Since typical VCSELs are in the order of 25 micrometers long, and have an operational power threshold below 1 mA, multiple VCSELs can be powered from a single power source.

In one embodiment, the VCSELs are one millimeter or less in height and are embedded in the casing material of body 12. Arrays of VCSELs, spaced by one-half inch, may be “sandwiched” between the polymer materials and interconnected by flat wire connector 106 in the device. The arrays of VCSELs may be electrically connected in parallel to avoid having a single inoperative VCSEL prevent numerous other VCSELs from operating.

In another embodiment, the light source includes one or more light emitting diodes (LEDs), such as hyper-luminescent red (“hyper-red”) LEDs. These LEDs can emit a light beam having 2000-3000 candle power and a wavelength of approximately 850 nm. The hyper-red LEDs may also be arrayed in a manner similar to the VCSELs in device 10. In still other embodiments, a combination of LEDs and VCSELs may be used wherein the hyper-red LEDs and the VCSELS are arrayed in an alternating fashion with several VCSELs for every hyper-red LED.

Operation of the power source 110 and programmable controller 112 may be initiated by means of a switch, such as a single-pole, double-throw or pressure switch. The programmable controller 112 may be configured with code for controlling the operation and timing of light sources 105. As used herein, timing control includes enabling the operation of the light source for the duration of the emission period, and disabling the operation of the light source for the duration of the interval period, in accordance with the prescribed light regimen. The programmable controller may have a 24 hour timing function which adjusts the operation of the light source for the emission and interval periods of time over the course of the 24 hour period.

The programmable controller may further include an oscillator to supply pulses at regular intervals (e.g., at one second intervals) to a counter/timer circuit. The counter circuit counts the pulses while a count decode logic circuit monitors the count. The count decode logic circuit is a multipurpose logic circuit which may comprise, for example, a PAL (programmable array logic) or a PLA (programmable logic array) that may be programmed to detect specified counts based on the emission periods and interval periods of the light regimen. For example, a count of 14,400 which would correspond to four hours of time and a count of 120 which would correspond to two minutes of time. Programmable controller 112 would be capable of maintaining the stored timing program without power being applied thereto. Upon detecting the programmed count, the count decode logic circuit outputs a laser enable pulse which enables laser current regulator circuits which regulate the power to each laser or LED of the light source. The regulator circuits, which are known in the art and which compare the current with a known voltage reference in order to maintain a constant current output, receive a voltage reference input from a voltage reference circuit. The voltage reference circuit may comprise an active band gap zener diode which supplies a constant voltage output (on the order of 1.2 to 1.5 volts) regardless of the voltage of the battery. At the same time, the logic circuit provides a RESET pulse to the counter/timer circuit to reset the count, and the counter continues counting the pulses from the oscillator. The laser enable pulse remains active for the duration of the emission period of the light regimen.

When power source 110 includes a rechargeable battery, the programmable controller 112 may also be configured to connect power source 110 to an external power source to recharge the battery. In some embodiments, power source 110 may be further coupled to a dedicated battery charge controller that is configured to monitor charging of the rechargeable battery and disconnect the battery from the external power source when a threshold charge level is reached.

Power source 110 may be selected so that it is capable of providing sufficient power for a defined duration of the laser therapy (e.g., one day, one week, etc.). A low battery voltage protection circuit may be included in programmable controller 112 to regulate the power supplied by the power source 110 and provide a desired voltage output (e.g., between 3.6 and 4.8 volts). The protection circuit may cease the supply of the power if the battery voltage drops below the threshold level (e.g., 3.6 volts) to avoid damage to circuit components. The power supplied by the power source 110 via the protection circuit is used to power the circuit components as well as the VCSELs and/or hyper-red LEDs. A standard LED driver circuit, as known in the art, may be used to drive the hyper-red LEDs.

In some embodiments, device 10 may include a heat sink coupled to each light source to address excess heat generated by the light source during the device operation. The heat sinks may be formed of copper and can be used to disburse the excess heat produced by the laser diode light source into the surrounding housing material. The heat sinks may also lend added structural integrity to the locations of the laser diodes. However, when VCSELs are used as the light source, the heat sinks may not be required due to low heat generated by the VCSELs.

FIG. 3 illustrates an example external light-generating device for mobilizing CD34+ with contoured body configured to fit a patient's body contour, such as the body contour of the patient's abdomen area.

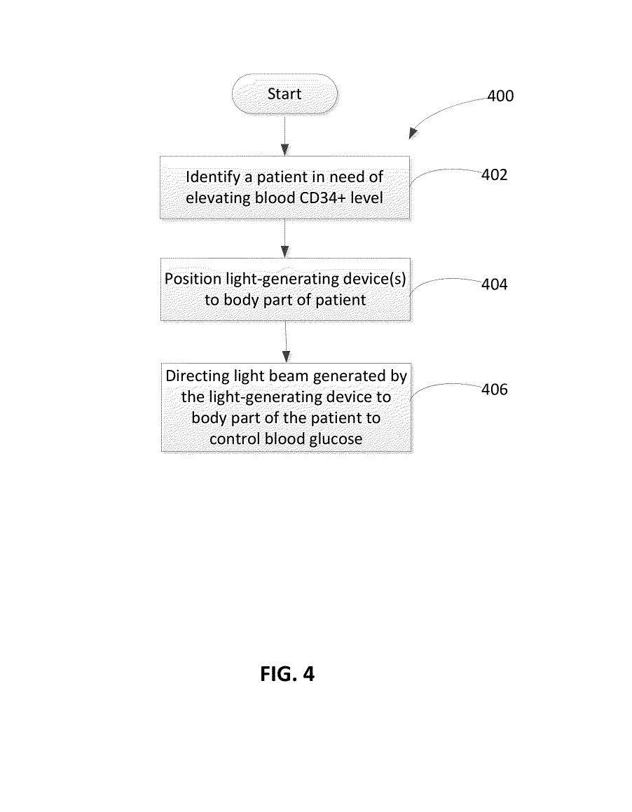

Now turning to FIG. 4, an example method 400 of elevating peripheral blood CD34+ cell count using a light generating device, such as the devices illustrated in FIGS. 1 to 3, is provided. It should be understood one or more steps of the method may be eliminated and/or sequence of which may be altered without affecting the effectiveness of the present invention.

At 402, the method includes identifying a patient in need of elevated CD34+ stem/progenitor cell count. For example, the patient may be a diabetic patient who has impaired ability to mobilize CD34+ cells and has a clinical condition such as myocardial infarction, stroke, diabetic foot ulcer, diabetic retinopathy that an elevated blood CD34+ stem/progenitor cell count in blood will help to improve the clinical outcome. In another example, the patient may be an elderly patient having a clinical condition that can benefit from an elevated CD34+ cell count. In a further example, the patient may be a donor.

At 404, the method includes positioning the light-generating device to a target area (e.g., stomach area, thigh area, etc.) of the patient's body. In various embodiments, the target area of the patient's body may be rich in CD34+ stem/progenitor cells and/or stromal precursor cells that give rise to CD34+ stem/progenitor cells. In various embodiments, the target area of the patient's body is rich in adipose tissue such as abdomen, buttock, and arm of the patient.

In various embodiments, the device is attached externally to the body part, such as a body part rich CD34+ stem/progenitor cells and/or stromal precursor cells that give rise to CD34+ stem/progenitor cells, such as a body part rich in adipose tissue such as a visceral adipose area of the body (that is, a visceral area with underlying adipose tissue), buttock, and arm of the patient. The device may be a portable device attached to the patient's body part. By using a portable and non-invasive device to provide the laser therapy, the treatment can be undergone without adversely affecting the patient's quality of life. The light-generating device may be attached to the selected part of the patient's body by the healthcare provider (e.g., physician) using a strap or bandage, so as to position the device over the desired treatment location. The strap or bandage may be sterilized, disposable, and made of a stretchable or flexible material (such as nylon) to allow the device to be properly attached to the patient's body while applying a positive biasing force to hold the device in place. Once placed upon the patient's body, the device may essentially look like a “patch”. This improves the wearability of the device. The patch can be worn by the patient for 12 hours per day, or 12 hours at night, and recharged when not worn. Further, since different light regimens can be provided for the patient using the same device (by changing the details on the programmable controller), patients may not need to replace the device frequently. For example, the patient may need a new patch only once every two years.

In various embodiments, the device is implanted internal to the body part, such as a body part rich CD34+ stem/progenitor cells and/or stromal precursor cells that give rise to CD34+ stem/progenitor cells, such as a body part rich in adipose tissue such as a visceral adipose area of the body (that is, a visceral area with underlying adipose tissue), buttock, and arm of the patient. In one example, the device is injected into the subcutaneous space of the body part. By using an implanted device implanted, the patient does not have to worry about remembering to wear the device and the treatment can be carried out with assurance of patient compliance.

At 406, the method includes selectively enabling the device according to the prescribed light regimen to elevate blood CD34+ cells in the peripheral blood of the patient. Operation of the device may be initiated by operating a switch. The light sources of the device may be configured to provide a visible feedback to the user (e.g., by flashing once) to confirm that the device is operational. The healthcare provider may operate the switch upon attaching the device to the patient's body. After the switch is operated and the device is attached to the desired area, the prescribed light regimen can begin. The patient may have to wear the device with the light regimen enabled for an extended period of time such as for one or more days, one or more weeks, or one or more months. The light directed to the body part may be on and off according to the prescribed light regimen. In various embodiment the CD34+ stem/progenitor cells in the peripheral blood of the patient is elevated. In various embodiments the CD34+ endothelial stem/progenitor cells in the peripheral blood is elevated preferentially as opposed to CD34+ hematopoietic stem/progenitor cells.

Optionally, a lag period may be provided between the operation of the switch and the actual initiation of the light regimen to allow sufficient time for the device to be properly positioned on the patient's body prior to initiation of the laser therapy. In embodiments where the device is an external device, the switch may be activated upon the prior activation of the proximity sensor, ensuring that the light regimen is initiated only when the device is placed on the patient's body.

After being positioned to the patient's body, the patient simply wears the device for the prescribed period of time and is free to conduct themselves in the normal course. The device automatically delivers the light therapy according to the prescribed light regimen, as indicated in the programmable controller, while the patient goes about their normal routine. With such a device, a single doctor visit replaces time consuming and costly, multiple office visits. A physician can initiate the light treatment regimen using the light-generating device and allow the patient to leave with the device attached to the body for a defined duration, such as one week of laser therapy, without the need for the patient to return to the physician's office. An appointment can be scheduled after that duration for the physician to determine the success of the light therapy and adjust (or reprogram) the light regimen, for example, based on measured CD34+ cell level in the peripheral blood. In various embodiments, the CD34+ cell level is CD34+ stem/progenitor cell level in peripheral blood of the patient. In various embodiments, the light regimen is adjusted based on a sub-population of CD34+ cell in the peripheral blood. In various embodiments, the light regimen is adjusted based on CD34+ endothelial stem/progenitor cell level in the peripheral blood of the patient. As such, since the device provides precisely timed laser treatments to target areas, the efficiency of the laser treatment is optimized and an overall length of the required treatment can be reduced.

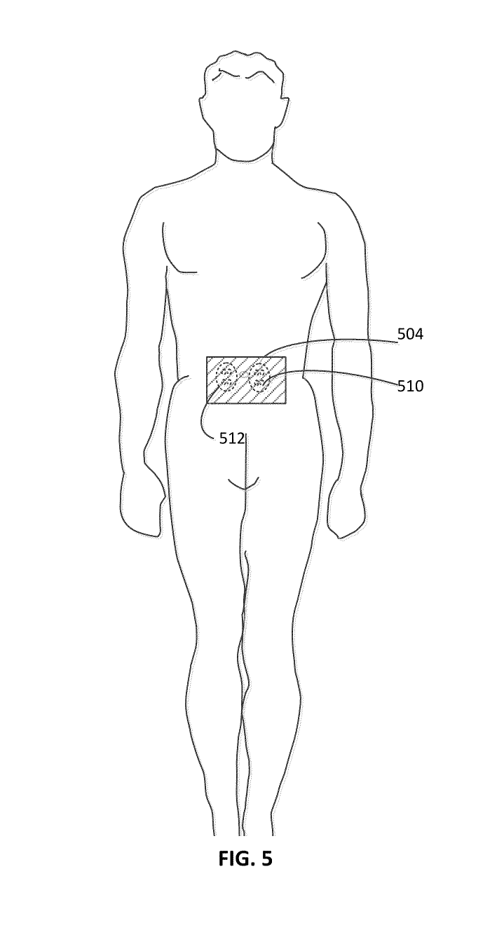

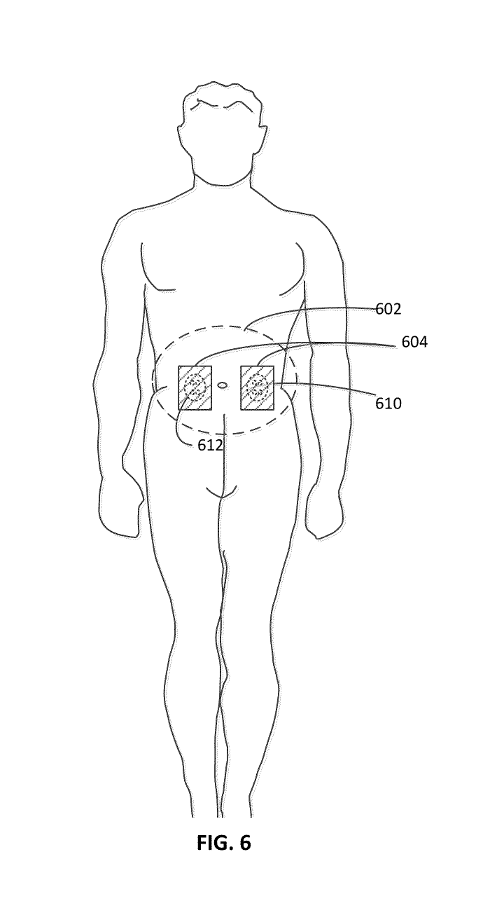

FIGS. 5 and 6 illustrate example embodiments for elevating CD34+ cell level in peripheral blood using a light generating device where the device is attached to a stomach area 302 (or visceral adipose area) of the patient's body.

Specifically, in FIG. 5, a plurality of light-generating devices 510, 512 (herein, two devices) are positioned along a waist circumference, specifically at the level of the navel, to target the low power laser light to the adipose tissue underlying the waist circumference. In the depicted example, each device 510, 512 include four lasers (dotted circles). The devices 510, 512 are kept in place with bandage 504.

In FIG. 6, the plurality of light-generating devices is arranged on the sides of the stomach area 602. Specifically, the plurality of devices are distributed into a left side device 610 that targets the low power laser light on the left side of the stomach area 602, and a right side device 612 that targets the low power laser light on the right side of the stomach area 602. The devices 610, 612 are kept in place with bandage 604.

In both FIGS. 5-6, light beams from the light-generating devices are focused on the patient's visceral adipose tissue. Since the visceral adipose tissue is rich in CD34+ stem/progenitor cells and precursor cells that give rise to CD34+ stem/progenitor cells, focusing the light beams on this region is most effective in elevating peripheral blood CD34+ cell level by increase CD34+ production and mobilization in the CD34+ cells in the adipose tissue.

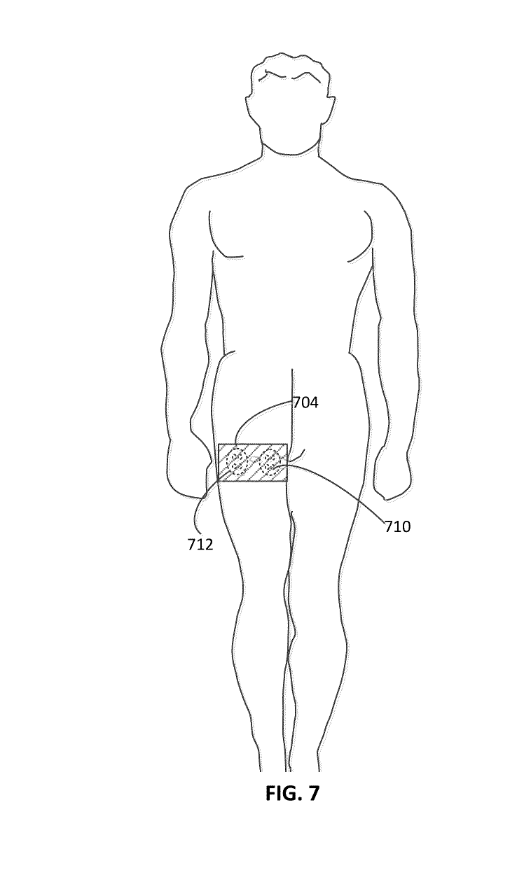

FIG. 7 illustrates another example embodiment for elevating peripheral blood CD34+ cell level. Specifically, in FIG. 7, the plurality of light-generating devices 710, 712 are positioned along a front portion of the patient's thigh area 702, using bandage 704, to target the low power laser light to the adipose tissue underlying the thigh and upper leg area. While FIG. 7 shows the plurality of devices aligned along a front portion of the thigh area, it will be appreciated that the devices may be alternatively be positioned on left and right thigh regions, or front and back regions of the thigh area, similar to the positioning shown in FIG. 6.

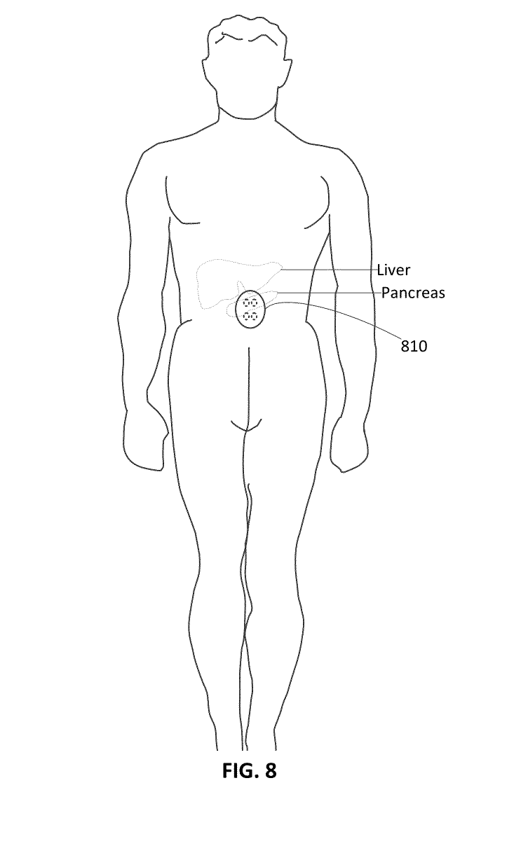

FIG. 8 illustrates an example embodiment of elevating CD34+ cell level in peripheral blood using a light generating device where the device 810 is implanted in the adipose tissue near the pancreas of the patient. The device 810 is oriented in such a way that the light beams generated by the light generating device are directed to the adipose tissue of this area according to a prescribe regimen.

In one example light regimen, the patient may wear two light-generating devices, each device having 4 vertical-cavity surface-emitting laser (VCSEL) 850 nm lasers at the light source that emit light beams over the underlying adipose tissue of the user for 10-14 hours per day. In each device, two of the four lasers may be selectively enabled to dose 9-12 mW of light energy (generated using 15 mA current) on the target area for an emission period of two minutes before being disabled. Next, the other two lasers of the device may be selectively enabled to dose 1-12 mW of light energy on the target area for an emission period of two minutes before being disabled. The lasers of the devices may be selectively disabled for an interval period of sixteen minutes. As such, the emission period followed by the interval period constitutes a cycle which is then repeated over the duration of the regimen. That is, at the end of the sixteen minutes, the lasers may be re-enabled for two minutes durations. In one example, the patient may wear the device for 14 hours during which the cycle (with emission periods separated by interval periods) is constantly repeated. At the end of the 14 hours, the device may be recharged. In one example, where the power source of the device is rechargeable (e.g., lithium ion battery), the power source is recharged for 5 hours after the 14 hour regimen, each day. It should be noted, the above described light regimen is an example light regimen. Other light regimen may be possible within the scope of the invention, for example, other light wavelength may be used, different light intensity may be used to achieve similar results, the emission period and resting period of the light sources may be adjusted, for example emission period may be 1 to 30 minutes and the resting period may be 1 to 60 minutes.

In one real example, an 80 year old patient having an initial CD34 count of 0% of gated cell population wore a light-generating device for elevating peripheral blood CD34+ level. The device includes 4 vertical-cavity surface-emitting laser (VCSEL) 850 nm lasers at the light source that emit light beams over the abdominal adipose tissue of the user for 10-14 hours per day. Each of the laser lights emits 10 mW of light energy (generated using 15 mA current). The light regimen consists of repeating cycle of 4 minutes on followed by 16 minutes off. After wearing the device for 1 month, the peripheral blood CD34+ level of the patient increased to 1% of gated cell population. The CD34+ cell levels were detected by CD45 gating method developed by Quest Diagnostics Nichols Institute, San Juan Capistrano.

In various embodiments, application of the light regimen to a patient is continued until predetermined criteria have been reached. In some embodiments, the light regimen is continued until the peripheral CD34+ cell level of the patient has reached a predetermined level. In some embodiments, the light regimen is continued until a predetermined time period (e.g., 1 month) has elapsed.

It should be noted, the above described light regimen is an example light regimen. Other light regimen may be possible within the scope of the invention, for example, other light wavelength may be used, different light intensity may be used to achieve similar results, the emission period and resting period of the light sources may be adjusted, for example emission period may be 1 to 30 minutes and the resting period may be 1 to 60 minutes.

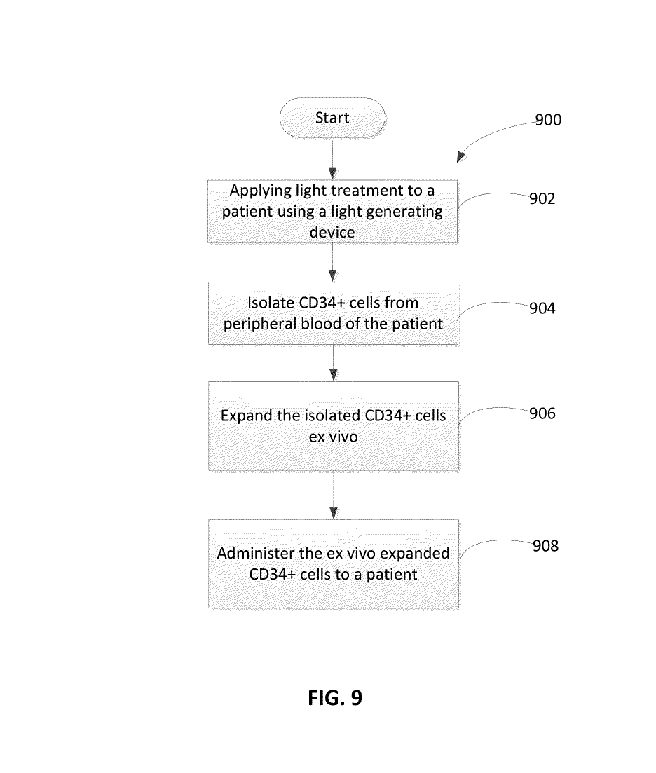

FIG. 9 illustrates an example embodiment of ex vivo expansion and administration of CD34+ cell level obtained from peripheral blood of a patient whose blood CD34+ cell count has been elevated using a light generating device described herein.

At 902, the method includes applying light regimen described herein using a light generating device described herein to elevate his/her blood CD34+ cell count. The various embodiments of the light generating device are described in reference to FIGS. 1-7. The various embodiments of the light regimen are described in reference to FIG. 8.

At 904, the method includes isolating CD34+ cells from the peripheral blood of the patient at the end of light regimen. The patient may be placed on such a light regimen for one or more week, one or more months, or one or more years. There are various ways to isolate CD34+ cells from the peripheral blood and is not limited to what is described below.

In some embodiments, heparinized blood sample is obtained from the patient. Mononuclear cells (MNCs) are isolated from the blood sample using density gradient centrifugation apheresis process. The obtained mononuclear cells are then incubated with a monoclonal antibody specific for the CD34 antigen. The cells treated with the monoclonal antibody are then loaded onto an immune-affinity column, such as avidin-immunoaffinity column. The column binds to the monoclonal antibodies and consequently also to the CD 34+ cells attached to the monoclonal antibodies. The bound CD 34+ cells are later removed from the immune-affinity column and suspended in a suitable medium to produce an enriched pool of CD34+ cells.

In some embodiments, monoclonal antibodies specific for the CD 34 antigen are directly bound to a solid phase (e.g. small beads). Mononuclear cells obtained through the apheresis process are incubated with the solid phase to allow the CD34+ cells to bind to the solid phase. After incubation, the solid phase with the bound CD34+ cells are separated from the rest of the mononuclear cells. The CD34+ cells are then released from the solid phase and suspended in a suitable medium to produce an enriched pool of CD34+ cells.

In some embodiments, CD 34+ cells are enriched from the peripheral blood by using a fluorescence activated cell sorter which is commercially available from for example Becton Dickinson. Mobilized peripheral blood progenitor cells are reacted with an anti-CD 34-antibody having a fluorochrome label. With the help of the fluorescence activated cell sorter it is possible to separate the cells and to obtain the CD 34+ cells.

At 906, the isolated CD34+ cells are expanded ex vivo. In some embodiments, the enriched CD 34+ cells are subsequently cultured and expanded in a suitable culturing medium, such as supplemented RPMI 1640 medium containing 10% fetal calf serum (Paesel, Germany). The culture medium may also contain heparinized autologous plasma, preferably in a concentration of about 1%. One of more growth factors are added to the culture medium, example growth factors include interleukin-1 (as described by Gery I. et. al., Method Enzymol. 116, 456-467 (1985); Lachmann et al., Methods Enzymol. 116, 467-497 (1985); March et al., Nature 315, 641 (1985)), interleukin-3 (as described in EPA 138 133, Ihle et al., Methods Enzymol. 116, 540-552 (1985); Otsuka et al., J. Immunol. 140, 2288-2295 (1988)), interleukin-6 (IL-6) (as described in Brakenhoff et al., J. Immunol. 139, 4116-4121 (1987), Brakenhoff et al., J. Immunol. 143, 1175-1182 (1989)), erythropoietin (EPO) (as described by Jacobs et al., Nature 313, 806-810 (1985), Sasaki et al., Methods Enzymol. 147, 328-340 (1987)), stem cell factor (SCF) (as described in WO 91/05 797, Nocka et al., EMBO J. 9, 3287-3294 (1990)) and interferon-γ (IFN-γ) (as described in EP 77 670, Gray et al., Nature 295, 503-508 (1982); Devos et al., Nucl. Acids Res. 10, 2487-2501 (1982); Yip et al., PNAS 79, 1820-1824 (1982) and Braude, Methods Enzymol. 119, 193-199 (1986)).

At 908, the ex vivo expanded CD34+ cells are administered back to a patient to treat one or more disease conditions of the patient such as myocardial infarction, stroke, diabetic ulcer, diabetic retinopathy. In some embodiments, the patient is the patient from whom the CD34+ cells were originally isolated. In some embodiments, the patient is a different individual from the patient from whom the CD34+ cells were isolated. In some embodiments, CD34+ cells such as ex vivo expanded CD 34+ cells are administered to a patient to repair and heal tissue damages. In some embodiments, ex vivo expanded CD34+ endothelial stem/progenitor cells are administered back to a patient.

As provided herein, the term “administering”, “administer” or “administration” with respect to delivery of cells, particularly CD34+ stem/progenitor cells, to a patient refers to injecting one or a plurality of cells with a syringe, inserting the cells with a catheter or surgically implanting the cells. In some embodiments, the cells are administered into a body cavity fluidly connected to a target tissue. In other embodiments, the cells are inserted using a syringe or catheter, or surgically implanted directly at the target tissue site. In some embodiments, the cells are administered systemically (e.g., parentally). In other embodiments, the cells are administered by intraocular delivery, intramuscular delivery, subcutaneous delivery or intraperitoneal delivery.

In should be noted that the term “patient” is meant to broadly include any animal. For example, the animal can be a mammal, a bird, a fish, a reptile, a fish, an insect or any other animal. Some non-limiting examples of mammals may include humans and other primates, equines such as horses, bovines such as cows, mice, rats, rabbits, Guinea Pigs, pigs, and the like.

The subject matter of the present disclosure includes all novel and nonobvious combinations and sub-combinations of the various processes, systems, devices, composition of matters, and configurations, and other features, functions, acts and/or properties disclosed herein, as well as any and all equivalents thereof.

The disclosed embodiments and various details are merely illustrative and are not to be construed as limiting and the invention can be implemented in various alternative ways. Further, the scope of the invention is limited only by the claims and the invention encompasses numerous alternatives, modifications and equivalents.

What is claimed:

1. A method for elevating peripheral blood CD34+ cell level of a patient using a light generating device, comprising:
positioning the light generating device to a body part of the patient; and
directing a beam of light generated by the light generating device to the body part of the patient to increase the peripheral blood CD34+ cell level of the patient.

2. The method of claim 1, wherein the light beam includes a wavelength of 400 to 1300 nm.

3. The method of claim 1, wherein the light beam is a low intensity beam generated by a power of 1-12 mW.

4. The method of claim 1, wherein the body part includes one body area selected from the group consisting of an adipose tissue rich area: abdominal, thigh, and buttocks.

5. The method of claim 1, wherein positioning the light generating device includes placing the light generating device over the body part but external to the patient body.

6. The method of claim 1, wherein positioning the light generating device includes implanting the light generating device to the body part so that the device is internal to the patient body.

7. The method of claim 1, wherein the light beam is generated by a light emitting diode.

8. The method of claim 1, wherein the light beam is generated by a laser diode.

9. The method of claim 1, wherein light beam is focused by a focusing lens.

10. The method of claim 1, wherein directing the light beam to the body part of the patient is according to a prescribed light regimen.

11. The method of claim 10, wherein the light regimen includes a plurality of emission periods during which the low power light is directed towards the body part, each of the plurality of emission periods separated from each other by a resting interval period during which no low power light is generated.

12. A light generating device for elevating peripheral blood CD34+ cell of a patient, comprising:
a source configured to generate a light beam;
a controller configured to direct a light beam generated to a body part of the patient to elevate the peripheral blood CD34+ cell level of the patient when the light generating device is positioned to the body part of the patient.

13. The device of claim 12, wherein the light beam have wavelength in the range of 400 to 1300 nm.

14. The device of claim 12, wherein the light beam is a low intensity beam generated by a power of 1-12 mW.

15. The device of claim 12, wherein the body part includes on body area selected from the group consisting of an adipose tissue rich area: abdominal, thigh, and buttocks.

16. The device of claim 12, wherein the source includes a light emitting diode for generating the light beam.

17. The device of claim 12, wherein the source includes a laser diode for generating the light beam.

18. The device of claim 12, wherein the light generating device includes a focusing lens for focusing the light beam.

19. The device of claim 12, wherein directing the light beam to the body part of the patient is according to a prescribed light regimen.

20. The device of claim 19, wherein the light regimen includes a plurality of emission periods during which the low power light is directed towards the body part, each of the plurality of emission periods separated from each other by a resting interval period during which no low power light is generated.

21. The device of claim 20, wherein each of the plurality of emission periods is 1-4 minutes in length and the resting interval period is 1 to 6 minute in length.
